(19) United States
(12) Patent Application Publication
(10) Pub. No.: US 2026/0029350 A1
(43) Pub. Date: Jan. 29, 2026

(54) METHOD FOR PREPARING ULTRAVIOLET (UV)-DEGRADABLE AND FUNCTIONALIZED CELLULOSE PAPER-BASED COLORIMETRIC SENSOR, AND APPLICATION THEREOF

(71) Applicants: JIANGSU UNIVERSITY, Zhenjiang (CN); Hunan Agricultural University, Changsha (CN)

(72) Inventors: Qin OUYANG, Zhenjiang (CN); Yu WANG, Zhenjiang (CN); Fei GAO, Zhenjiang (CN); Zhonghua LIU, Changsha (CN)

(21) Appl. No.: 19/344,655

(22) Filed: Sep. 30, 2025

(30) Foreign Application Priority Data
Oct. 10, 2024 (CN) 202411409828.2

(51) Int. Cl. G01N 21/80; D21H 17/13; D21H 17/66; D21H 25/02; D21H 25/06; D21H 27/08; G01N 21/01; G01N 33/14

(52) U.S. Cl. CPC G01N 21/80; D21H 17/13; D21H 17/66; D21H 25/02; D21H 25/06; D21H 27/08; G01N 21/01; G01N 33/14; G01N 2021/0181

(57) ABSTRACT

A method for preparing an ultraviolet (UV)-degradable and functionalized cellulose paper-based colorimetric sensor, in which a TiO₂-loaded cellulose filter paper is prepared, from which a TiO₂/OTS-loaded functionalized cellulose filter paper is prepared; and the TiO₂/OTS-loaded functionalized cellulose filter paper is combined with colorimetric materials to obtain the UV-degradable and functionalized cellulose paper-based colorimetric sensor with multiple hydrophilic dye-loading regions and a hydrophobic isolation region. This application further provides a food quality evaluation method, in which a food quality evaluation model is established based on the UV-degradable and functionalized cellulose paper-based colorimetric sensor.

### CROSS-REFERENCE TO RELATED APPLICATIONS

This application claims the benefit of priority from Chinese Patent Application No. 202411409828.2, filed on Oct. 10, 2024. The content of the aforementioned application, including any intervening amendments made thereto, is incorporated herein by reference in its entirety.

### TECHNICAL FIELD

This application relates to colorimetric sensing detection technology and non-destructive detection technology for food and agricultural products, and more particularly to a method for preparing an ultraviolet (UV)-degradable and functionalized cellulose paper-based colorimetric sensor, and an application thereof.

### BACKGROUND

Volatile organic compounds in food refer to a class of chemical substances that can volatilize into the air and have an important impact on food quality. Monitoring these volatile organic compounds in food enables the evaluation of food quality and provides timely warnings of potential quality issues during food processing or storage. By promptly monitoring and controlling volatile components in food, the flavor characteristics and product quality of food can be ensured, thereby enhancing consumer satisfaction and market competitiveness of products. Therefore, monitoring volatile components in food is of great significance.

Gas chromatography (GC) or gas chromatography-mass spectrometry (GC-MS) are conventional methods for detecting volatile compounds. However, these techniques involve complex sample preparation and high detection costs, making them unsuitable for on-site testing. Colorimetric sensing detection technology for volatile compounds has been developed based on the olfactory perception mechanism of mammals. It offers rapid response, low cost, and high portability, and has been widely used in the detection of volatile substances. At present, colorimetric sensors still face certain problems in practical applications. Paper-based materials, as common flexible materials for preparing colorimetric sensors, are favored by researchers due to their ease of use, good biocompatibility, and low cost. However, the inherent hydrophilicity of filter paper can lead to drift in colorimetric response signals, significantly hindering its practical application. Additionally, strong capillary forces between colorimetric units cause uneven diffusion of solutions, resulting in mutual interference among sensing units and poor fabrication stability of colorimetric sensors. Colorimetric dyes such as metalloporphyrins, boron-dipyrromethenes, and pH indicators are the core substances used for constructing sensors, but their use can cause environmental pollution. With the development of society and the growing awareness of environmental protection, developing degradable substrates for constructing colorimetric sensors can avoid secondary pollution and has significant importance for environmental protection.

### SUMMARY

An object of the disclosure is to provide a paper-based material of functionalized cellulose filter paper for addressing the problems of poor stability and low moisture resistance of colorimetric sensors. The paper-based material includes a hydrophilic colorimetric dye loading regions and a hydrophobic isolation region, where the large-area hydrophobic region can effectively prevent the filter paper from absorbing moisture from the environment, thereby enabling the prepared colorimetric sensor to exhibit enhanced moisture resistance. At the same time, the hydrophobic isolation region can effectively prevent mutual interference among colorimetric units, thereby enabling the prepared colorimetric sensor to exhibit improved stability. Another object of the disclosure is to provide a paper-based material having TiO₂ photocatalytic properties for addressing the problem of potential environmental pollution. When the paper-based material is used for preparing the colorimetric sensor, the dye on the colorimetric sensor can be degraded merely by ultraviolet irradiation, thereby enabling the prepared colorimetric sensor to exhibit better environmental compatibility. The paper-based colorimetric sensor of the disclosure thus solves the technical problems existing in the prior art, including high detection cost, poor repeatability, low moisture resistance, complicated detection processes, and environmental pollution caused by dyes.

Technical solutions of the present disclosure are described as follows.

In a first aspect, this application provides a method for preparing an ultraviolet (UV)-degradable and functionalized cellulose paper-based colorimetric sensor, comprising:
(1) preparing a TiO₂-loaded cellulose filter paper through steps of:
(1a) obtaining a filter paper according to a desired size by cutting followed by immersion in absolute ethanol (to clean and activate hydroxyl groups on a surface of a cellulose structure) and drying to obtain a preliminarily-treated filter paper, wherein the filter paper is a cellulose filter paper;
(1b) mixing absolute ethanol, tetrabutyl titanate and glacial acetic acid to obtain a mixed solution;
(1c) immersing the preliminarily-treated filter paper in the mixed solution followed by shaking on a shaker and drying, and repeating steps of immersing in the mixed solution, shaking and drying several times to obtain a secondarily-treated filter paper; and
(1d) subjecting the secondarily-treated filter paper to hydrolysis in deionized water to obtain the TiO₂-loaded cellulose filter paper;
(2) preparing a TiO₂/octadecyltrichlorosilane (OTS)-loaded functionalized filter paper through steps of:
immersing the TiO₂-loaded cellulose filter paper obtained in step (1) in an OTS-n-hexane mixed solution followed by washing with n-hexane and absolute ethanol and drying to obtain a TiO₂/OTS-loaded cellulose filter paper; and
covering the TiO₂/OTS-loaded cellulose filter paper with a cover plate followed by irradiation with a UV lamp and washing with absolute ethanol to obtain the TiO₂/OTS-loaded functionalized cellulose filter paper, wherein the cover plate is made of glass, the cover plate has the same area as the TiO₂/OTS-loaded cellulose filter paper, a plurality of circular holes are provided evenly spaced apart on the cover plate, and the TiO₂/OTS-loaded functionalized cellulose filter paper has a plurality of circular hydrophilic colorimetric dye loading regions and a hydrophobic isolation region; and
(3) preparing a colorimetric material solution;
dropwise adding the colorimetric material solution to the plurality of circular hydrophilic colorimetric dye loading regions of the TiO₂/OTS-loaded functionalized cellulose filter paper prepared in step (2), so as to obtain the UV-degradable and functionalized cellulose paper-based colorimetric sensor.

In some embodiments, in step (1a), the filter paper is a qualitative filter paper having a size of 30-40 mm×30-40 mm; and the filter paper is immersed in the absolute ethanol for 3-6 h and dried at 30-50° C. for 20 min or less.

In some embodiments, in step (1b), a volume ratio of the absolute ethanol to the tetrabutyl titanate to the glacial acetic acid is 10:3:1; in step (1c), the shaking is carried out at a speed of 180-200 r/min for 30-60 min, the drying is performed at 30-50° C. for 20 min or less, and the steps of immersing in the mixed solution, shaking and drying are repeated 3-5 times; and in step (1d), the hydrolysis is carried out at 85-95° C. for 2-5 h.

In some embodiments, in step (2), a volume ratio of OTS to n-hexane in the OTS-n-hexane mixed solution is 1:1000; the TiO₂-loaded cellulose filter paper is immersed in the OTS-n-hexane mixed solution for 5-10 min; the step of washing with n-hexane and absolute ethanol is repeated 3-5 times; and the drying is performed at 30-50° C. for 20 min or less.

In some embodiments, in step (2), the cover plate is a rectangular cuboid having a length of 39 mm, a width of 39 mm and a thickness of 4 mm; a distance between centers of adjacent circular holes of the plurality of circular holes is 9 mm; and each of the plurality of circular holes has a diameter of 6 mm.

In some embodiments, the UV lamp is a dual-wavelength lamp having wavelengths of 185 nm and 254 nm; the cover plate is provided below the UV lamp; and a distance between the UV lamp and the cover plate is 1-2 cm; the irradiating is performed for 40-60 min; after UV irradiation, the hydrophobic isolation region is formed at an area of the TiO₂/OTS-loaded cellulose filter paper covered by the cover plate; and OTS in regions of the TiO₂/OTS-loaded cellulose filter paper exposed through the plurality of circular holes is decomposed under the UV irradiation, so as to form the plurality of circular hydrophilic colorimetric dye loading regions each with a diameter of 6 mm on the TiO₂/OTS-loaded cellulose filter paper.

In some embodiments, X colorimetric material solutions are prepared, and X is a positive integer;
the X colorimetric material solutions are each independently composed of a first solution, a second solution or a combination thereof; wherein the first solution is a solution of a metalloporphyrin or boron-dipyrromethene in dichloromethane, and the second solution is a solution of a pH indicator in ethanol; and
a ratio of the metalloporphyrin or the boron-dipyrromethene to the dichloromethane in the first solution is 2 mg:1 mL; and a ratio of the pH indicator to the ethanol in the second solution is 2 mg:1 mL.

In some embodiments, the pH indicator is selected from the group consisting of bromothymol blue, bromocresol green, methyl red, bromophenol blue, cresol red and mauveine; and
the metalloporphyrin is manganese tetraphenylporphyrin; and
the boron-dipyrromethene is 8-(4-methoxyphenyl)-4,4-difluoro-2,6-dibromo-boron-dipyrromethene.

In some embodiments, an amount of each of the X colorimetric material solutions applied onto a corresponding one of the plurality of circular hydrophilic colorimetric dye loading regions of the TiO₂/OTS-loaded functionalized cellulose filter paper is 1.5-2 μL.

In a second aspect, this application provides a method for monitoring food quality, comprising:
(1) preparing a UV-degradable and functionalized cellulose paper-based colorimetric sensor according to the method described above;
(b) establishing a food quality evaluation model through steps of:
(b1) selecting a plurality of food samples varying in quality grade, wherein different quality grades correspond to different volatile odor compounds, and the different volatile odor compounds induce different color changes in the UV-degradable and functionalized cellulose paper-based colorimetric sensor; and
(b2) capturing an image of the UV-degradable and functionalized cellulose-based colorimetric sensor before reaction using a camera;
respectively placing the plurality of food samples and the UV-degradable and functionalized cellulose-based colorimetric sensor in a reaction container in a sealed state for a period of time to reaction between volatile odor compounds from the plurality of food samples and the UV-degradable and functionalized cellulose-based colorimetric sensor;
capturing an image of the UV-degradable and functionalized cellulose-based colorimetric sensor after reaction using the camera followed by storage in a computer;
determining, by the computer, positions of colorimetric units in the image of the UV-degradable and functionalized cellulose-based colorimetric sensor before reaction and the image of the UV-degradable and functionalized cellulose-based colorimetric sensor after reaction, extracting color features of each of the colorimetric units, and calculating a difference in mean gray values of each of the colorimetric units before and after reaction as a feature variable of each of the colorimetric units; and
combining feature variables of the plurality of food samples to form a feature matrix, and constructing a long short-term memory (LSTM) recurrent neural network model with the feature matrix as an input and a true quality grade of each of the plurality of food samples as a training label as the food quality evaluation model; and
(c) performing quality evaluation of a to-be-detected food sample through steps of:
obtaining a feature variable of the to-be-detected food sample according to steps (b1-b2); and
inputting the feature variable of the to-be-detected food sample into the food quality evaluation model to obtain quality grade of the to-be-detected food sample, so as to achieve quality evaluation of the to-be-detected food sample.

In some embodiments, in step (b1), the plurality of food samples comprise a tea sample.

In some embodiments, in step (b2), an amount of each of the plurality of food samples is 0.5-1.5 g, and the reaction in the reaction container is carried out for 10-30 min; and the UV-degradable and functionalized cellulose-based colorimetric sensor is fixed at a top of the reaction container.

In some embodiments, in step (b2), the feature variable of each of the colorimetric units is extracted through steps of:
locating a position of each of the colorimetric units on the UV-degradable and functionalized cellulose-based colorimetric sensor using the computer;
decomposing each of the image before reaction and the image after reaction into three single-channel images (R channel, G channel and B channel), and extracting hue (H), saturation(S), value (V), lightness (L), red-green value (a), and yellow-blue value (b) of each of the image before reaction and the image after reaction;
calculating differences between values of R, G, B, H, S, V, L, a, and b of each of the colorimetric units before and after reaction to obtain ΔR, ΔG, ΔB, ΔH, ΔS, ΔV, ΔL, Δa and Δb, respectively; and calculating a Euclidean distance (ED) based on ED=\sqrt{ΔR²+ΔG²+ΔB²};
wherein ΔR, ΔG, ΔB, ΔH, ΔS, ΔV, ΔL, Δa and Δb and ED are feature variables of a corresponding colorimetric unit, X colorimetric units yield Y feature variables, and Y=10×X; and wherein the number of the plurality of food samples for constructing the food quality evaluation model is N; N samples involve n treatment levels with m samples for each of the n treatment levels, and N=n×m, n is a positive integer equal to or larger than 2, and m and N are positive integers.

In some embodiments, in step (b2), the food quality evaluation model is constructed through steps of:
denoting the feature matrix as S with a size of N×Y, wherein Nis the number of the plurality of food samples, and Y is a total number of feature variables corresponding to X colorimetric units;
inputting the feature matrix S into the LSTM recurrent neural network model to generate a hidden state matrix H; and
selectively mapping the hidden state matrix H to an output matrix H′ through a fully connected layer of the LSTM recurrent neural network model, wherein H′=f(Wₕ×H+bₕ), f is an activation function, Wₕ is a weight matrix, and bn is a bias term (Model parameters of the LSTM recurrent neural network model are optimized by minimizing prediction errors between the output matrix H′ and actual labels. The model parameters include weight matrices within the LSTM recurrent neural network model, and the weight matrix Wₕ and the bias term bn of the fully connected layer; and the LSTM recurrent neural network model for food quality evaluation is constructed with a true sample-quality matrix T serving as a training label.

In some embodiments, in step (c), the quality evaluation is performed through steps of:
obtaining Y feature variables of M to-be-detected samples according to step (b), so as to form a feature variable matrix R, wherein R has a size of M×Y; and
inputting the feature variable matrix R into the LSTM recurrent neural network model to generate an output Q corresponding to quality grade information of the M to-be-detected samples, so as to achieve quality evaluation.

Compared to the prior art, the present disclosure has the following beneficial effects.
(1) The present disclosure provides a method for preparing an UV-degradable and functionalized cellulose paper-based colorimetric sensor and a method for food quality evaluation, effectively addressing technical challenges in the prior art, including high detection costs, poor sensor stability, complex detection procedures, and environmental pollution caused by dyes, and enabling rapid evaluation of food quality based on volatile components.
(2) The cellulose filter paper is employed as a substrate, a TiO₂ is loaded on a surface of the cellulose filter paper to prepare a photocatalytic layer, enabling the colorimetric dyes to undergo photocatalytic degradation under ultraviolet light, thereby enhancing the environmental compatibility of the colorimetric sensor and significantly improving the modification efficiency of the cellulose filter paper.
(3) In the present disclosure, a plurality of hydrophilic colorimetric dye loading regions are formed on the surface of the cellulose filter paper through OTS modification combined with ultraviolet irradiation, each hydrophilic colorimetric dye loading region is isolated by the hydrophobic isolation region, such that the hydrophilic colorimetric dye loading regions do not interfere with each other, thereby enhancing the fabrication stability and moisture resistance of the paper-based sensor.
(4) A long short-term memory (LSTM) recurrent neural network model characterized by its inherent temporal nature is established for rapid food quality evaluation. The model takes signals collected by the colorimetric sensor as an input and food processing levels as an output, providing a generalizable evaluation framework that enhances efficiency and is significant for assessing food quality during processing.

### BRIEF DESCRIPTION OF THE DRAWINGS

FIG. 1A is a scanning electron microscopy (SEM) image of a TiO₂-loaded cellulose filter paper;

FIG. 1B is an energy-dispersive X-ray spectroscopy (EDS) spectrum of the TiO₂-loaded cellulose filter paper;

FIG. 2 is a diagram illustrating optimization of octadecyltrichlorosilane (OTS) modification time on a surface of the TiO₂-loaded cellulose filter paper;

FIG. 3A is a SEM image of a TiO₂/OTS-loaded cellulose filter paper;

FIGS. 3B-C are EDS spectra of the TiO₂/OTS-loaded cellulose filter paper;

FIG. 4A is a schematic diagram illustrating a glass cover plate and ultraviolet irradiation;

FIG. 4B is a schematic diagram showing hydrophilic dye loading regions and a hydrophobic isolation region formed after ultraviolet irradiation;

FIG. 5 is a diagram illustrating optimization of functional modification time for a TiO₂/OTS-loaded functionalized cellulose filter paper;

FIG. 6 is a comparative diagram showing colorimetric sensors fabricated from the TiO₂/OTS-loaded functionalized cellulose filter paper and from an ordinary filter paper;

FIG. 7A is a diagram showing moisture resistance of colorimetric sensors prepared from the ordinary filter paper;

FIG. 7B is a diagram showing moisture resistance of colorimetric sensors prepared from the TiO₂/OTS-loaded functionalized cellulose filter paper;

FIGS. 8A-B illustrate long short-term memory (LSTM) classification results of tea leaves with varying degrees of withering in Example 2 of the present disclosure, where A corresponds to a training set and B corresponds to a prediction set; and

FIGS. 9A-B are diagrams showing ultraviolet degradation efficiency of an ultraviolet-degradable and functionalized cellulose paper-based colorimetric sensor prepared in Example 2 of the present disclosure.

### DETAILED DESCRIPTION OF EMBODIMENTS

Various exemplary embodiments of the present disclosure will be described in detail herein. Such detailed descriptions should not be construed as a limitation on the present disclosure but rather as a more specific explanation of certain aspects, features and implementations thereof.

It should be understood that the terminology used herein is for the purpose of describing particular embodiments only and is not intended to be limiting of the present disclosure. Unless otherwise stated, all technical and scientific terms used herein have the same meaning as commonly understood by those skilled in the art relevant to the present disclosure.

While preferred methods and materials are described, any similar or equivalent methods and materials may also be used in the implementation or testing of the present disclosure. All references cited herein are incorporated by reference to disclose and describe methods and/or materials related to those references. In case of any conflict, the content of this specification shall prevail.

Various modifications and variations of the embodiments described herein can be made without departing from the scope or spirit of the present disclosure, which will be apparent to those skilled in the art.

Other embodiments obtained from the present disclosure are also obvious to those skilled in the art. The description and embodiments provided herein are merely illustrative.

### Example 1

Provided herein was a method for preparing an ultraviolet (UV)-degradable and functionalized cellulose paper-based colorimetric sensor, including the following steps.

Step (1) A TiO₂-loaded cellulose filter paper was prepared through the following steps.

A qualitative filter paper was obtained by cutting into a size of 40×40 mm and immersed in absolute ethanol for 3 h to clean and activate hydroxyl groups on a surface of a cellulose structure, and dried in an oven at 50° C. for 15 min to obtain a preliminarily-treated filter paper, where the qualitative filter paper is a cellulose filter paper.

Absolute ethanol, tetrabutyl titanate, and glacial acetic acid were mixed at a volume ratio of 10:3:1 to prepare a first mixed solution. The preliminarily-treated filter paper was immersed in the first mixed solution, subjected to shaking on a shaker at a speed of 190 r/min for 30 min, and dried in the oven at 50° C. for 15 min. The steps of immersing in the first mixed solution, shaking and drying were repeated three times, such that the cellulose filter paper was sufficiently impregnated with the tetrabutyl titanate, so as to obtain a secondarily-treated filter paper.

The secondarily-treated filter paper was subjected to hydrolysis in ultrapure water at 95° C. for 5 h to obtain the TiO₂-loaded cellulose filter paper.

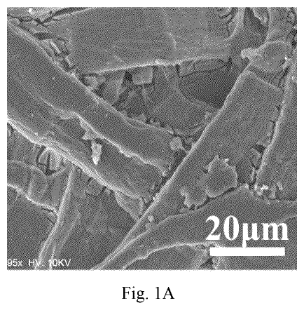

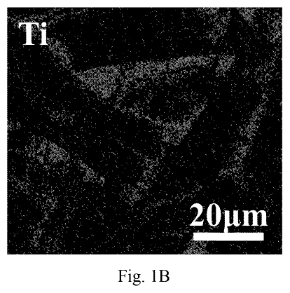

FIG. 1A was a scanning electron microscopy (SEM) image of the TiO₂-loaded cellulose filter paper. FIG. 1B was an energy-dispersive X-ray spectroscopy (EDS) spectrum of the TiO₂-loaded cellulose filter paper. As shown in FIG. 1A, surfaces and pores of the filter paper fibers were observed to be filled with substances. The EDS spectrum in FIG. 1B indicated that the substances filling the surfaces and pores of the filter paper fibers were TiO₂. These results indicated that TiO₂ had been successfully loaded on a surface of the cellulose filter paper.

Step (2) A TiO₂/octadecyltrichlorosilane (OTS)-loaded functionalized filter paper was prepared through the following steps.

OTS was mixed with n-hexane to obtain a second mixed solution, in which a volume ratio of OTS to n-hexane in the was 1:1000.

The TiO₂-loaded cellulose filter paper was immersed in the second mixed solution for 5 min, washed three times with n-hexane and absolute ethanol, and dried at room temperature to obtain a TiO₂/OTS-loaded cellulose filter paper.

The TiO₂/OTS-loaded cellulose filter paper was covered with a cover plate, irradiated with a UV lamp at a distance of 2 cm for 30 min and washed with absolute ethanol to obtain the TiO₂/OTS-loaded functionalized cellulose filter paper, where the cover plate was made of glass, the UV lamp was a dual-wavelength lamp having wavelengths of 185 nm and 254 nm, and the TiO₂/OTS-loaded functionalized cellulose filter paper had a plurality of circular hydrophilic colorimetric dye loading regions and a hydrophobic isolation region.

The cover plate was a rectangular cuboid having a length of 39 mm, a width of 39 mm and a thickness of 4 mm. A plurality of circular holes are provided evenly spaced apart on the cover plate. A distance between centers of adjacent circular holes was 9 mm. A diameter of each circular hole was 6 mm. The plurality of circular holes were located within the cover plate without intersecting any edges. For any circular hole adjacent to a periphery of the cover plate, a distance from a center of such circular hole to a nearest edge of the cover plate was 6 mm.

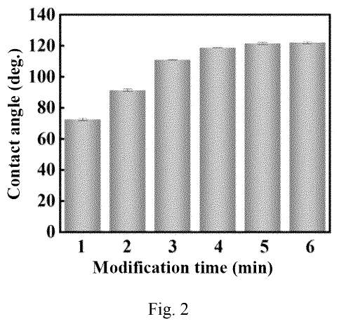

FIG. 2 was a diagram illustrating optimization of OTS modification time on a surface of the TiO₂-loaded cellulose filter paper. As shown in FIG. 2, with the extension of the treatment time, a surface tension of the TiO₂-loaded cellulose filter paper decreased while a contact angle increased. When the treatment time reached 5 min, the surface tension and the contact angle of the TiO₂-loaded cellulose filter paper had essentially stabilized, indicating that the optimal time for OTS modification of the TiO₂-loaded cellulose filter paper surface was 5 min.

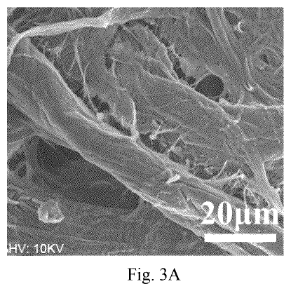

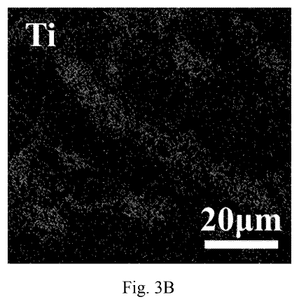

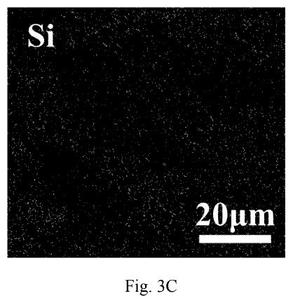

FIG. 3A was a SEM image of the TiO₂/OTS-loaded cellulose filter paper. FIGS. 3B-C were EDS spectra of the TiO₂/OTS-loaded cellulose filter paper. As shown in FIG. 3A, numerous chemical substances had grown on the surfaces and within the pores of the filter paper fibers. The EDS spectra in FIGS. 3B-3C indicated that the substances filling the surfaces and pores of the filter paper fibers were TiO₂ and OTS. These results demonstrated that TiO₂ and OTS had been successfully loaded on the surface of the cellulose filter paper, demonstrating successful hydrophobic modification of the filter paper.

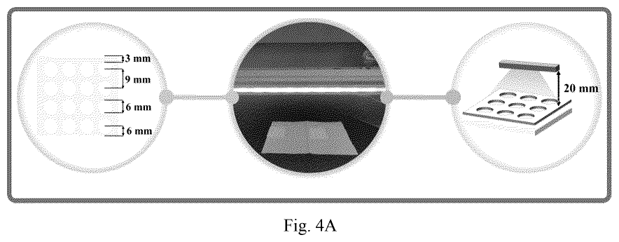

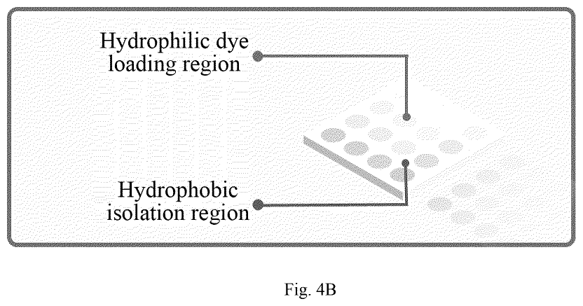

FIG. 4A was a schematic diagram illustrating the cover plate and UV irradiation, and FIG. 4B was a schematic diagram showing hydrophilic dye loading regions and a hydrophobic isolation region formed after UV irradiation. As shown in FIG. 4A, the arrangement and diameters of the circular holes on the cover plate and a distance between the UV lamp and the cover plate during irradiation was observed. As shown in FIG. 4B, the TiO₂/OTS-loaded cellulose filter paper was covered with the cover plate, and after UV irradiation, the TiO₂/OTS-loaded functionalized cellulose filter paper having circular hydrophilic colorimetric dye loading regions and the hydrophobic isolation region was produced.

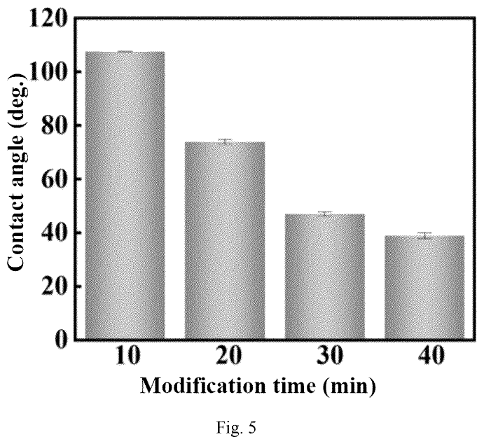

FIG. 5 was a diagram illustrating optimization of functional modification time for the TiO₂/OTS-loaded functionalized cellulose filter paper. As shown in FIG. 5, with the extension of UV irradiation time, a surface tension of the TiO₂/OTS-loaded functionalized cellulose filter paper increased, while the contact angle decreased. When UV irradiation reached 30 min, the surface tension and the contact angle of the TiO₂/OTS-loaded functionalized cellulose filter paper had essentially stabilized, indicating that the optimal time for functional modification of the TiO₂/OTS-loaded functionalized cellulose filter paper was 30 min.

Step (3) Eight colorimetric solutions were prepared, consisting of a solution of a boron-dipyrromethene in dichloromethane, a solution of a metalloporphyrin in dichloromethane, and six ethanol solutions of pH indicators.

The eight colorimetric solutions were denoted as a first colorimetric solution (S1), a second colorimetric solution (S2), a third colorimetric solution (S3), a fourth colorimetric solution (S4), a fifth colorimetric solution (S5), a sixth colorimetric solution (S6), a seventh colorimetric solution (S7) and an eighth colorimetric solution (S8).

The first colorimetric solution was a solution of 8-(4-methoxyphenyl)-4,4-difluoro-2,6-dibromo-boron-dipyrromethene in dichloromethane, where a ratio of the 8-(4-methoxyphenyl)-4,4-difluoro-2,6-dibromo-boron-dipyrromethene to the dichloromethane was 20 mg:10 mL.

The second colorimetric solution was a solution of manganese tetraphenylporphyrin in dichloromethane, where a ratio of the manganese tetraphenylporphyrin to the dichloromethane was 20 mg:10 mL.

The third colorimetric solution was a solution of bromothymol blue in ethanol, where a ratio of the bromothymol blue to the ethanol was 20 mg:10 mL.

The fourth colorimetric solution was a solution of bromocresol green in ethanol, where a ratio of the bromocresol green to the ethanol was 20 mg:10 mL.

The fifth colorimetric solution was a solution of methyl red in ethanol, where a ratio of the methyl red to the ethanol was 20 mg:10 mL.

The sixth colorimetric solution was a solution of bromophenol blue in ethanol, where a ratio of the bromophenol blue to the ethanol was 20 mg:10 mL.

The seventh colorimetric solution was a solution of cresol red in ethanol, where a ratio of the cresol red to the ethanol was 20 mg:10 mL.

The eighth colorimetric solution was a solution of mauveine in ethanol, where a ratio of the mauveine to the ethanol was 20 mg:10 mL.

1.5 μL of each of the eight colorimetric solutions was drawn using a micropipette and deposited onto a corresponding one of the hydrophilic colorimetric dye loading regions of the TiO₂/OTS-loaded functionalized cellulose filter paper, resulting in a colorimetric sensor fabricated from the TiO₂/OTS-loaded functionalized cellulose filter paper, namely the UV-degradable and functionalized cellulose paper-based colorimetric sensor.

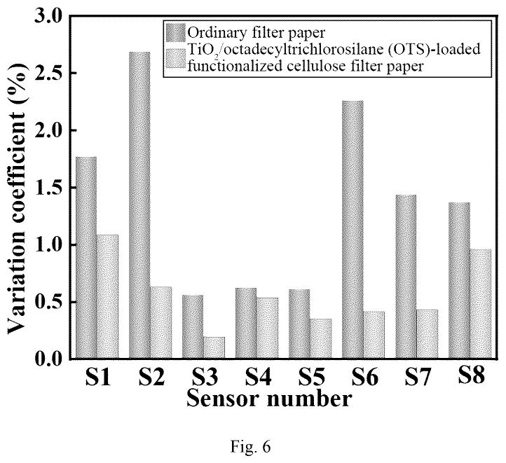

FIG. 6 was a comparative diagram showing colorimetric sensors fabricated from the TiO₂/OTS-loaded functionalized cellulose filter paper and from an ordinary filter paper. As shown in FIG. 6, the colorimetric sensors prepared from the TiO₂/OTS-loaded functionalized cellulose filter paper exhibited lower coefficients of variation, indicating that the colorimetric sensors prepared from the TiO₂/OTS-loaded functionalized cellulose filter paper had higher stability.

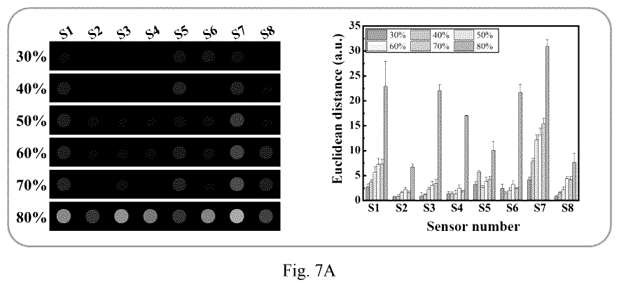

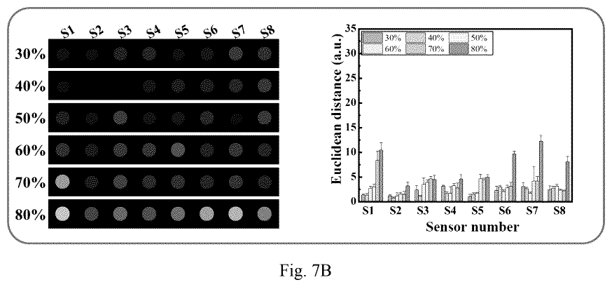

FIG. 7A was a diagram showing moisture resistance of colorimetric sensors prepared from the ordinary filter paper. FIG. 7B was a diagram showing moisture resistance of colorimetric sensors prepared from the TiO₂/OTS-loaded functionalized cellulose filter paper. As shown in FIGS. 7A-B, under the same humidity conditions, the response values of the colorimetric sensors prepared from the TiO₂/OTS-loaded functionalized cellulose filter paper were lower, indicating that the colorimetric sensors prepared from the TiO₂/OTS-loaded functionalized cellulose filter paper exhibited stronger moisture resistance.

### Example 2

Provided herein was a method for evaluating the quality of withered tea leaves using a UV-degradable and functionalized cellulose paper-based colorimetric sensor (abbreviated as “cellulose paper-based colorimetric sensor” in this embodiment), including the following steps.

### Step (1) Selection of Samples

Samples having time sequence were selected as tea leaves with different withering times, the tea leaves having been grown in Jurong City, Jiangsu Province. The degree of withering of the tea leaves was classified into grades 1 to 7 according to the withering time.

### Grade Descriptions

A first grade: The sample was fresh leaves after plucking, with a moisture content of about 70%. The leaves had high water content, were hard and brittle, and emitted a grassy aroma.

A second grade: The sample was fresh leaves after plucking and subjected to far-infrared irradiation for 3 h. The moisture content was reduced, the flexibility of the leaves increased, and the aroma richness was enhanced.

A third grade: The sample was the second-grade sample after natural moisture evaporation for 3 h, with a total withering duration of 6 h. The moisture further dissipated, the leaves became soft and not brittle, the aroma richness was enhanced, and the grassy aroma weakened.

A fourth grade: The sample was the third-grade sample after natural moisture evaporation for 3 h, with a total withering duration of 9 h. The moisture further dissipated, the leaves became soft and not brittle, the aroma richness was enhanced, the grassy aroma weakened, and floral-fruity aroma appeared.

A fifth grade: The sample was the fourth-grade sample after natural moisture evaporation for 3 h, with a total withering duration of 12 h. The moisture further dissipated, the leaves became soft and not brittle, the grassy aroma weakened, and the floral-fruity aroma appeared.

A sixth grade: The sample was the fifth-grade sample after natural moisture evaporation for 3 h, with a total withering duration of 15 h. The moisture further dissipated, the leaves became soft and not brittle, the grassy aroma weakened, and the floral-fruity aroma became more pronounced.

A seventh grade: The sample was the sixth-grade sample after natural moisture evaporation for 3 h, with a total withering duration of 18 h, and the moisture content of the sample was about 60%. The moisture further dissipated, the leaves became soft and not brittle, the grassy aroma weakened, and the floral-fruity aroma further enhanced.

Step (2) An image of the cellulose paper-based colorimetric sensor before reaction was captured using a camera. Subsequently, 0.6 g of each of the above withered tea leaf samples at different withering degrees was weighed and placed together with the cellulose paper-based colorimetric sensor in a reaction vessel. The prepared cellulose paper-based colorimetric sensor was fixed at a top of the reaction vessel. The cellulose paper-based colorimetric sensor was allowed to fully react with volatile substances of each of the above withered tea leaf samples at different withering degrees at 25° C. for 20 min. Finally, an image of the cellulose paper-based colorimetric sensor after reaction was captured using the camera and stored in a computer.

Step (3) Positions of colorimetric units in the image of the cellulose paper-based colorimetric sensor before reaction and the image of the cellulose paper-based colorimetric sensor after reaction were determined using the computer. Each of the image before reaction and the image after reaction was decomposed into three single-channel images (R channel, G channel and B channel), and hue (H), saturation(S), value (V), lightness (L), red-green value (a), and yellow-blue value (b) of each of the image before reaction and the image after reaction was extracted. Differences between values of R, G, B, H, S, V, L, a, and b of each of the colorimetric units before and after reaction was calculated to obtain ΔR, ΔG, ΔB, ΔH, ΔS, ΔV, ΔL, Δa and Δb. A Euclidean distance (ED) based on ED=\sqrt{ΔR²+ΔG²+ΔB²} was calculated. ΔR, ΔG, ΔB, ΔH, ΔS, ΔV, ΔL, Δa and Δb and ED were feature variables of a corresponding colorimetric unit, and a total of 80 feature variables were obtained from eight colorimetric units. For the seven withering grades of tea leaves, each grade contained 25 samples, resulting in 175 samples in total. The 80 feature variables of the 175 samples were combined to obtain a feature matrix S. A long short-term memory (LSTM) recurrent neural network model for evaluating the quality of withered tea leaves was constructed using the feature matrix S as an input and the corresponding true quality grade matrix T of the samples as a training label.

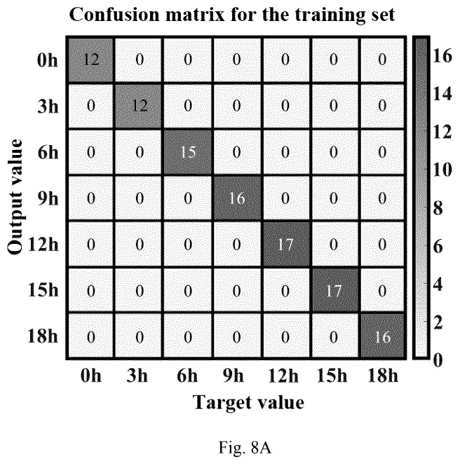

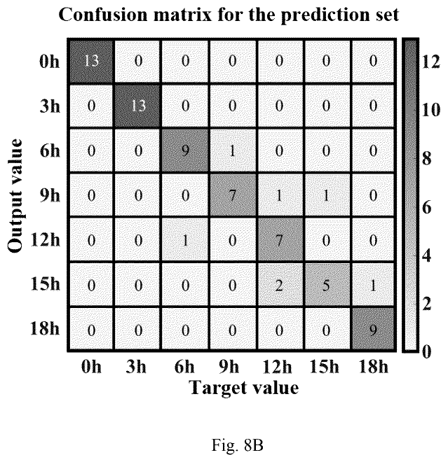

FIG. 8A was a LSTM recurrent neural network model for evaluating the quality of withered tea leaves, which was established based on sample information collected by the cellulose paper-based colorimetric sensor. The training accuracy of the LSTM recurrent neural network model had reached 100%.

Step (4) Seventy tea samples with unknown grades were taken, and 80 feature variables for the 70 to-be-detected tea samples were obtained according to the methods described in steps (2) and (3), forming a feature variable matrix R (R had a size of 70×80). The LSTM recurrent neural network model constructed in step (3) was then invoked, and the feature variable matrix R was used as an input to generate an output matrix Q corresponding to quality grade information of the 70 to-be-detected tea samples, thereby achieving rapid evaluation of tea quality during the withering process.

FIG. 8B showed the results of predicting the quality of the 70 to-be-detected tea samples by using the LSTM recurrent neural network model constructed in step (3). The prediction accuracy had reached 90%, confirming that the cellulose paper-based colorimetric sensor constructed in the present disclosure could achieve rapid evaluation of tea quality during the withering process.

### Example 3

Provided herein was a UV degradation method for a UV-degradable and functionalized cellulose paper-based colorimetric sensor, including the following steps.

Step (1) A used UV-degradable and functionalized cellulose paper-based colorimetric sensor was irradiated under a UV lamp with dual-wavelengths of 185 nm and 254 nm at a distance of 2 cm to conduct a degradation experiment.

Step (2) An image of the UV-degradable and functionalized cellulose paper-based colorimetric sensor before the degradation experiment and an image of the UV-degradable and functionalized cellulose paper-based colorimetric sensor after the degradation experiment were captured using a camera. Positions of colorimetric units in the image of the UV-degradable and functionalized cellulose paper-based colorimetric sensor before the degradation experiment and the image of the UV-degradable and functionalized cellulose paper-based colorimetric sensor after the degradation experiment were determined using a computer. Each of the image before the degradation experiment and the image after the degradation experiment of each of the colorimetric units was decomposed into three single-channel images (R channel, G channel and B channel). Color information of each colorimetric unit before the degradation experiment was recorded as Ra, Ga and Bu, while the color information after the degradation experiment was recorded as Rb, Gb and Bb.

Subsequently, background information of the UV-degradable and functionalized cellulose paper-based colorimetric sensor was extracted. R channel, G channel and B channel information of the background was recorded as R₀, G₀ and B₀.

Step (3) A degradation rate of the UV-degradable and functionalized cellulose paper-based colorimetric sensor was calculated using the following equation:

$$Δ {R}_{a} = {R}_{0} - {R}_{a} ;$$

$$Δ {G}_{a} = {G}_{0} - {G}_{a} ;$$

$$Δ {B}_{a} = {B}_{0} - {B}_{a} ;$$

$${ED}_{a} = \sqrt{Δ {R}_{a}^{2} + Δ {G}_{a}^{2} + Δ {B}_{a}^{2}} ;$$

$$Δ {R}_{b} = {R}_{0} - {R}_{b} ;$$

$$Δ {G}_{b} = {G}_{0} - {G}_{b} ;$$

$$Δ {B}_{b} = {B}_{0} - {B}_{b} ;$$

$${ED}_{b} = \sqrt{Δ {R}_{b}^{2} + Δ {G}_{b}^{2} + Δ {B}_{b}^{2}} ; and$$

$$Degradation rate = \frac{E {D}_{a} - E {D}_{b}}{E {D}_{a}} \times 100 \% .$$

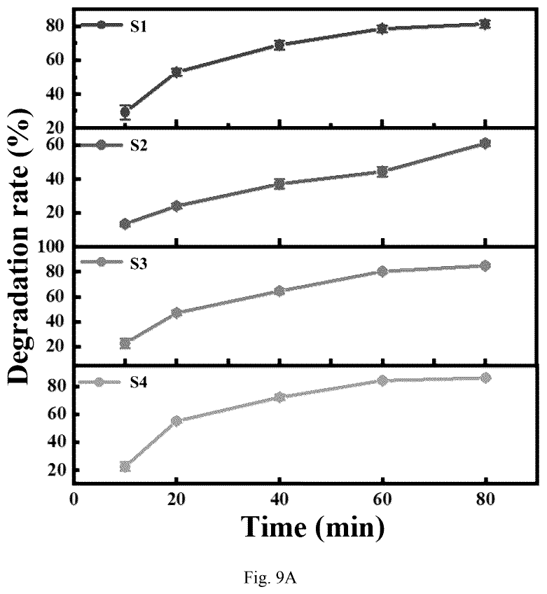

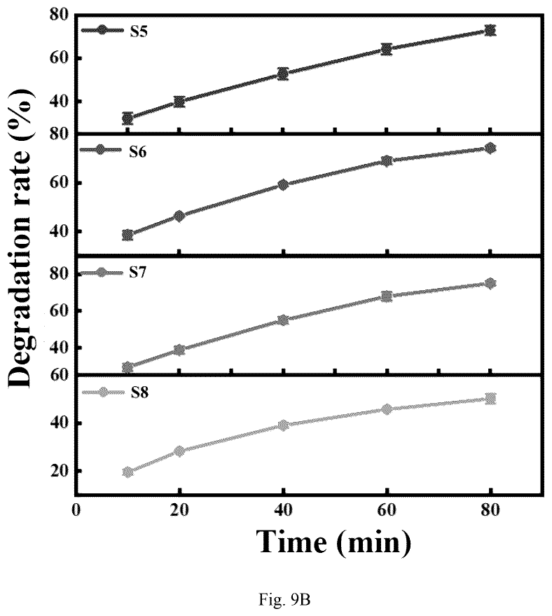

FIGS. 9A-B were diagrams showing UV degradation efficiency of the UV-degradable and functionalized cellulose paper-based colorimetric sensor prepared in the embodiments. As shown in FIGS. 9A-B, the degradation rate of each colorimetric unit in the sensor increased with the extension of irradiation time. When the irradiation time reached 80 min, the sensor achieved a degradation efficiency of 60-90%.

Described above are merely preferred embodiments of the present disclosure, and are not intended to limit the scope of the present disclosure. It should be understood that various modifications, changes and replacements made by those skilled in the art without departing from the spirit of the disclosure shall fall within the scope of the present disclosure defined by the appended claims.

What is claimed is:

1. A method for preparing an ultraviolet (UV)-degradable and functionalized cellulose paper-based colorimetric sensor, comprising:
(1) preparing a TiO₂-loaded cellulose filter paper through steps of:
(1a) obtaining a filter paper according to a desired size by cutting followed by immersion in absolute ethanol and drying to obtain a preliminarily-treated filter paper, wherein the filter paper is a cellulose filter paper;
(1b) mixing absolute ethanol, tetrabutyl titanate and glacial acetic acid to obtain a mixed solution;
(1c) immersing the preliminarily-treated filter paper in the mixed solution followed by shaking on a shaker and drying, and repeating steps of immersing in the mixed solution, shaking and drying several times to obtain a secondarily-treated filter paper; and
(1d) subjecting the secondarily-treated filter paper to hydrolysis in deionized water to obtain the TiO₂-loaded cellulose filter paper;
(2) preparing a TiO₂/octadecyltrichlorosilane (OTS)-loaded functionalized filter paper through steps of:
immersing the TiO₂-loaded cellulose filter paper obtained in step (1) in an OTS-n-hexane mixed solution followed by washing with n-hexane and absolute ethanol and drying to obtain a TiO₂/OTS-loaded cellulose filter paper; and
covering the TiO₂/OTS-loaded cellulose filter paper with a cover plate followed by irradiation with a UV lamp and washing with absolute ethanol to obtain the TiO₂/OTS-loaded functionalized cellulose filter paper, wherein the cover plate is made of glass, the cover plate has the same area as the TiO₂/OTS-loaded cellulose filter paper, a plurality of circular holes are provided evenly spaced apart on the cover plate, and the TiO₂/OTS-loaded functionalized cellulose filter paper has a plurality of circular hydrophilic colorimetric dye loading regions and a hydrophobic isolation region; and
(3) preparing a colorimetric material solution;
dropwise adding the colorimetric material solution to the plurality of circular hydrophilic colorimetric dye loading regions of the TiO₂/OTS-loaded functionalized cellulose filter paper prepared in step (2), so as to obtain the UV-degradable and functionalized cellulose paper-based colorimetric sensor.

2. The method of claim 1, wherein in step (1a), the filter paper is a qualitative filter paper having a size of 30-40 mm×30-40 mm; and the filter paper is immersed in the absolute ethanol for 3-6 h, and dried at 30-50° C. for 20 min or less;
in step (1b), a volume ratio of the absolute ethanol to the tetrabutyl titanate to the glacial acetic acid is 10:3:1;
in step (1c), the shaking is carried out at a speed of 180-200 r/min for 30-60 min, the drying is performed at 30-50° C. for 20 min or less, and the steps of immersing in the mixed solution, shaking and drying are repeated 3-5 times; and
in step (1d), the hydrolysis is carried out at 85-95° C. for 2-5 h.

3. The method of claim 1, wherein in step (2), a volume ratio of OTS to n-hexane in the OTS-n-hexane mixed solution is 1:1000;
the TiO₂-loaded cellulose filter paper is immersed in the OTS-n-hexane mixed solution for 5-10 min;
the step of washing with n-hexane and absolute ethanol is repeated 3-5 times;
the drying is performed at 30-50° C. for 20 min or less;
the cover plate is a rectangular cuboid having a length of 39 mm, a width of 39 mm and a thickness of 4 mm; a distance between centers of adjacent circular holes of the plurality of circular holes is 9 mm; and each of the plurality of circular holes has a diameter of 6 mm;
the UV lamp is a dual-wavelength lamp having wavelengths of 185 nm and 254 nm; the cover plate is provided below the UV lamp; and a distance between the UV lamp and the cover plate is 1-2 cm;
the irradiating is performed for 40-60 min; and
after UV irradiation, the hydrophobic isolation region is formed at an area of the TiO₂/OTS-loaded cellulose filter paper covered by the cover plate; and OTS in regions of the TiO₂/OTS-loaded cellulose filter paper exposed through the plurality of circular holes is decomposed under the UV irradiation, so as to form the plurality of circular hydrophilic colorimetric dye loading regions each with a diameter of 6 mm on the TiO₂/OTS-loaded cellulose filter paper.

4. The method of claim 1, wherein in step (3), X colorimetric material solutions are prepared, and X is a positive integer;
the X colorimetric material solutions are each independently composed of a first solution, a second solution or a combination thereof, wherein the first solution is a solution of a metalloporphyrin or boron-dipyrromethene in dichloromethane, and the second solution is a solution of a pH indicator in ethanol; and
a ratio of the metalloporphyrin or the boron-dipyrromethene to the dichloromethane in the first solution is 2 mg:1 mL; a ratio of the pH indicator to the ethanol in the second solution is 2 mg:1 mL; and an amount of each of the X colorimetric material solutions applied onto a corresponding one of the plurality of circular hydrophilic colorimetric dye loading regions of the TiO₂/OTS-loaded functionalized cellulose filter paper is 1.5-2 μL.

5. The method of claim 4, wherein in step (3), the pH indicator is selected from the group consisting of bromothymol blue, bromocresol green, methyl red, bromophenol blue, cresol red and mauveine; and
the metalloporphyrin is manganese tetraphenylporphyrin; and
the boron-dipyrromethene is 8-(4-methoxyphenyl)-4,4-difluoro-2,6-dibromo-boron-dipyrromethene.

6. A method for monitoring food quality, comprising:
(a) preparing a UV-degradable and functionalized cellulose paper-based colorimetric sensor according to the method of claim 1;
(b) establishing a food quality evaluation model through steps of:
(b1) selecting a plurality of food samples varying in quality grade, wherein different quality grades correspond to different volatile odor compounds, and the different volatile odor compounds induce different color changes in the UV-degradable and functionalized cellulose paper-based colorimetric sensor; and
(b2) capturing an image of the UV-degradable and functionalized cellulose-based colorimetric sensor before reaction using a camera;
respectively placing the plurality of food samples and the UV-degradable and functionalized cellulose-based colorimetric sensor in a reaction container in a sealed state for a period of time to allow reaction between volatile odor compounds from the plurality of food samples and the UV-degradable and functionalized cellulose-based colorimetric sensor;
capturing an image of the UV-degradable and functionalized cellulose-based colorimetric sensor after reaction using the camera followed by storage in a computer;
determining, by the computer, positions of colorimetric units in the image of the UV-degradable and functionalized cellulose-based colorimetric sensor before reaction and the image of the UV-degradable and functionalized cellulose-based colorimetric sensor after reaction, extracting color features of each of the colorimetric units, and calculating a difference in mean gray values of each of the colorimetric units before and after reaction as a feature variable of each of the colorimetric units; and
combining feature variables of the plurality of food samples to form a feature matrix, and constructing a long short-term memory (LSTM) recurrent neural network model with the feature matrix as an input and a true quality grade of each of the plurality of food samples as a training label as the food quality evaluation model; and
(c) performing quality evaluation of a to-be-detected food sample through steps of:
obtaining a feature variable of the to-be-detected food sample according to steps (b1-b2); and
inputting the feature variable of the to-be-detected food sample into the food quality evaluation model to obtain quality grade of the to-be-detected food sample, so as to achieve quality evaluation of the to-be-detected food sample.

7. The method of claim 6, wherein in step (b1), the plurality of food samples comprise a tea sample.

8. The method of claim 6, wherein in step (b2), an amount of each of the plurality of food samples is 0.5-1.5 g, and the reaction in the reaction container is carried out for 10-30 min; and
the UV-degradable and functionalized cellulose-based colorimetric sensor is fixed at a top of the reaction container.

9. The method of claim 6, wherein in step (b2), the feature variable of each of the colorimetric units is extracted through steps of:
locating a position of each of the colorimetric units on the UV-degradable and functionalized cellulose-based colorimetric sensor using the computer;
decomposing each of the image before reaction and the image after reaction into three single-channel images (R channel, G channel and B channel), and extracting hue (H), saturation(S), value (V), lightness (L), red-green value (a), and yellow-blue value (b) of each of the image before reaction and the image after reaction;
calculating differences between values of R, G, B, H, S, V, L, a, and b of each of the colorimetric units before and after reaction to obtain ΔR, ΔG, ΔB, ΔH, ΔS, ΔV, ΔL, Δa and Δb, respectively; and
calculating a Euclidean distance (ED) based on ED=\sqrt{ΔR²+ΔG²+ΔB²};
wherein ΔR, ΔG, ΔB, ΔH, ΔS, ΔV, ΔL, Δa and Δb and ED are feature variables of a corresponding colorimetric unit, X colorimetric units yield Y feature variables, and Y=10×X; and
wherein the number of the plurality of food samples for constructing the food quality evaluation model is N; N samples involve n treatment levels with m samples for each of the n treatment levels, and N=n×m, n is a positive integer equal to or larger than 2, and m and N are positive integers.

10. The method according to claim 6, wherein in step (b2), the food quality evaluation model is constructed through steps of:
denoting the feature matrix as S with a size of N×Y, wherein Nis the number of the plurality of food samples, and Y is a total number of feature variables corresponding to X colorimetric units;
inputting the feature matrix S into the LSTM recurrent neural network model to generate a hidden state matrix H; and
selectively mapping the hidden state matrix H to an output matrix H′ through a fully connected layer of the LSTM recurrent neural network model, wherein H′=f(Wₕ×H+bn), f is an activation function, Wₕ is a weight matrix, and bn is a bias term.

11. The method of claim 6, wherein in step (c), the quality evaluation is performed through steps of:
obtaining Y feature variables of M to-be-detected samples according to step (b), so as to form a feature variable matrix R, wherein R has a size of M×Y; and
inputting the feature variable matrix R into the LSTM recurrent neural network model to generate an output Q corresponding to quality grade information of the M to-be-detected samples, so as to achieve quality evaluation.
